(19) United States
(12) Patent Application Publication
(10) Pub. No.: US 2014/0031299 A1
(43) Pub. Date: Jan. 30, 2014

(54) NUTRITIONAL SUPPLEMENTS AND ASSOCIATED TREATMENT METHODS

(71) Applicant: Martin L. Pall, Portland, OR (US)

(72) Inventor: Martin L. Pall, Portland, OR (US)

(21) Appl. No.: 13/558,116

(22) Filed: Jul. 25, 2012

(51) Int. Cl. A61K 31/7004; A61P 29/00; A23L 1/29

(52) U.S. Cl. 514/23; 426/648

(57) ABSTRACT

A nutritional supplement, comprising a combination of resveratrol, nicotinic acid or a nicotinic acid precursor, and D-ribose in nutritionally effective amounts; and wherein the combination is configured to increase Sirtuin 1 enzymatic activity and to initiate increased NAD+ production for sustaining said Sirtuin 1 enzymatic activity. In some examples the nutritional supplement is configured to target a certain physiologic stress and is delivered in treatments including a resveratrol dose of 30 mg per day, a nicotinic acid or nicotinic acid precursor dose of 30 mg per day, and a D-ribose dose of 3 grams per day.

### BACKGROUND

The present disclosure relates generally to nutritional supplements. In particular, nutritional supplements utilizing unique combinations of resveratrol, nicotinic acid or nicotinic acid precursors, and the sugar D-ribose are described.

Nutritional supplements are used for many purposes. They can be added to the diet

to boost overall health and energy; to provide immune system support and reduce the risks of illness and age-related conditions; to improve performance in athletic and mental activities; and to support the healing process during illness and disease.

The nutritional ingredients in these supplements may include among other things: vitamins, minerals, herbs or other botanicals, amino acids, and substances such as enzymes, organ tissues, glandulars, and metabolites. Nutritional supplements can also be extracts or concentrates, and may be found in many forms such as tablets, capsules, softgels, gelcaps, liquids, powders, certain food stuff, bars, and pre-mixed drinks.

A particular subset of nutritional supplements includes a compound called resveratrol. Known nutritional supplements utilizing the phenol 3,5,4′-trihydroxystilbene (resveratrol), in either the trans form or a cis/trans mixture, are not entirely satisfactory for the range of applications in which they are employed. For example, existing nutritional supplements fail to address the depletion of the coenzyme nicotinamide adenine dinucleotide (NAD+) in the cellular environment.

Nucleotides are important constituents in a number of key biomolecules utilized in maintaining the health of living organisms. NAD+, a metabolite of ATP, is a coenzyme that has a vital role in organismal oxidation-reduction reactions.

Importantly, NAD+ formation is driven by the introduction of nicotinic acid or certain nicotinic acid precursors into the cellular environment of a given organism. These components, necessary for NAD+ synthesis, are provided by the vitamin Niacin.

Insufficient NAD+ levels negate most of the beneficial effects of resveratrol treatments. Existing nutritional supplements provide more resveratrol than can be reasonably utilized without simultaneously increasing NAD+ production. This means that much of the resveratrol supplied in the supplement remains unused and the benefits are unrealized.

Thus, there exists a need for nutritional supplements that improve upon and advance the design of known supplements by adequately addressing the shortfall of biomolecules relevant to resveratrol metabolism. Examples of new and useful supplement combinations relevant to the needs existing in the field are discussed below.

Disclosure addressing one or more of the identified existing needs is provided in the detailed description below. Examples of references relevant to resveratrol treatment include U.S. Patent Reference: Pub. No 2001/0056071. The complete disclosure of this publication is herein incorporated by reference for all purposes.

### SUMMARY

A nutritional supplement, comprising a combination of resveratrol, nicotinic acid or a nicotinic acid precursor, and D-ribose in nutritionally effective amounts; and wherein the combination is configured to increase Sirtuin 1 enzymatic activity and to initiate increased NAD+ production for sustaining said Sirtuin 1 enzymatic activity. In some examples the nutritional supplement is configured to target a certain physiologic stress and is delivered in treatments including a resveratrol dose of 30 mg per day, a nicotinic acid or nicotinic acid precursor dose of 30 mg per day, and a D-ribose dose of 3 grams per day.

### BRIEF DESCRIPTION OF THE DRAWINGS

FIG. 1 is a flow diagram of a biological process resulting from a first example of a nutritional supplement containing resveratrol, nicotinic acid or nicotinic acid precursor, and D-ribose in a capsule form.

FIG. 2 is a schematic diagram representing the nutritional supplement shown in FIG. 1 being introduced into an organism.

FIG. 3 is a schematic diagram of a second example of a nutritional supplement, where the components resveratrol, nicotinic acid or nicotinic acid precursor, and D-ribose are contained in separate preparations being introduced into a person.

FIG. 4 is a schematic diagram of a third example of a nutritional supplement, where the individual components resveratrol, nicotinic acid or nicotinic acid precursor, and D-ribose are contained in separate preparations being introduced into an organism.

### DETAILED DESCRIPTION

The disclosed nutritional supplements and associated treatment methods will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and treatment methods; however, for the sake of brevity, each and every contemplated variation is not individually described in the following detailed description.

Throughout the following detailed description, examples of various nutritional supplements and treatment methods are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

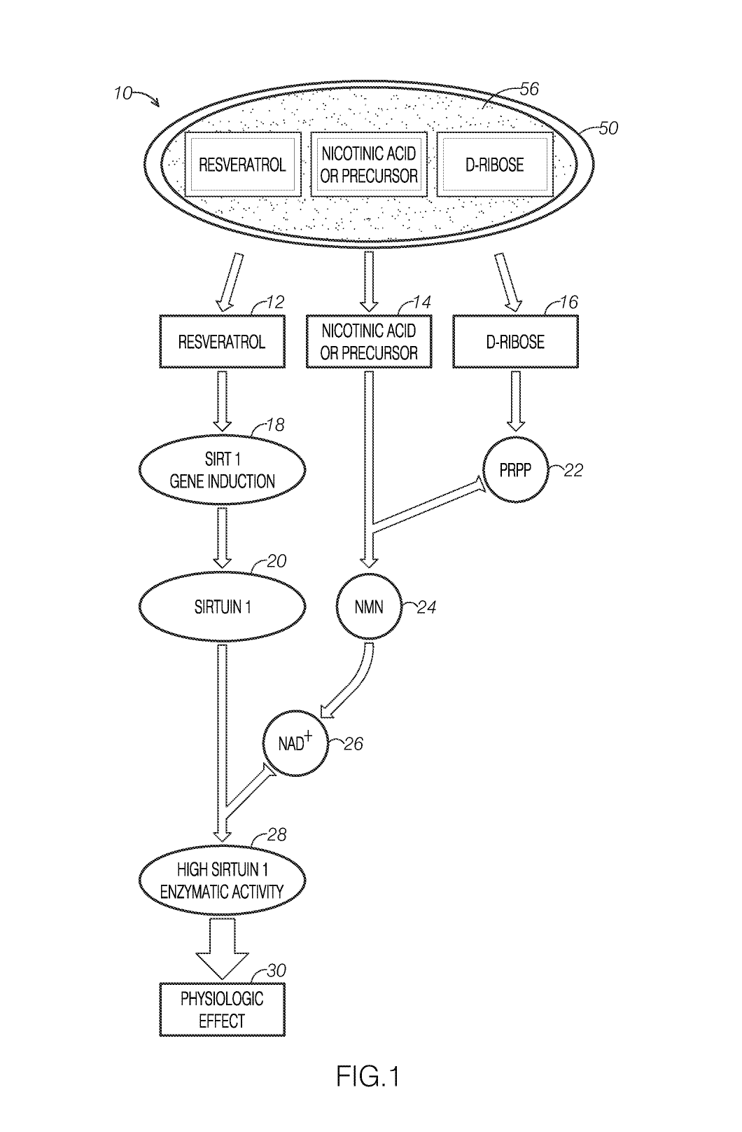

With reference to FIGS. 1-4, various examples of preparations and treatment methods involving a nutritional supplement, supplement 10, will now be described. Supplement 10 includes nutritionally effective amounts of resveratrol 12, nicotinic acid or nicotinic acid precursor 14, and D-ribose 16.

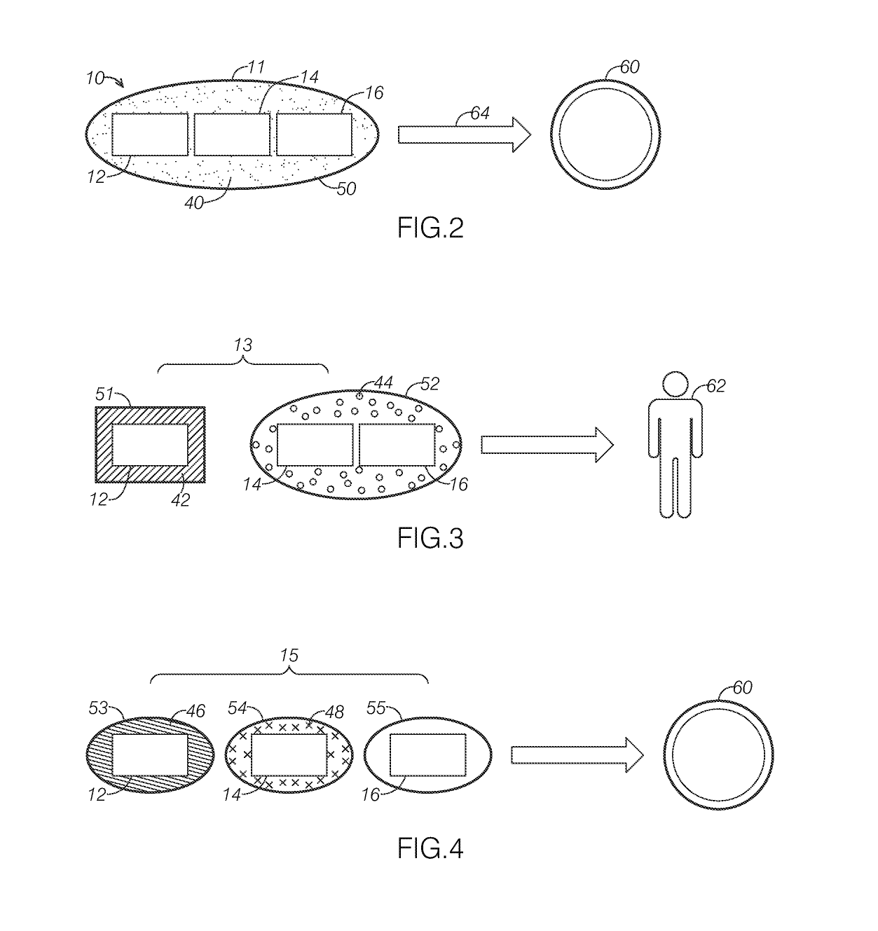

With particular reference to FIGS. 2-4, various preparation forms containing supplement 10 are described. The relevant preparations include a combination of supplement 10 in a single preparation form 11, a combination of supplement 10 introduced via a preparation pair 13, and a combination of supplement 10 where the individual components resveratrol 12, nicotinic acid or nicotinic acid precursor 14, and D-ribose 16, are introduced in separate preparations 15.

Supplement 10 functions to reduce the deleterious effects of physiologic stress on the health of living organisms. The novel combination comprising supplement 10 acts to provide effective amounts of key biomolecules for combatting physiologic stressors that cause chronic inflammation.

There are dozens of chronic inflammatory diseases characterized by elevated inflammatory cytokines and other inflammatory markers, but also by oxidative stress, elevated superoxide radical, elevated levels of the inducible nitric oxide synthase (iNOS), elevated levels of the potent oxidant peroxynitrite formed from nitric oxide and superoxide radical, dysfunction of mitochondria, elevated activity of the transcription factor NF-kappaB, depletion of the enzyme cofactor tetrahydrobiopterin and in the brain, at least, excitotoxicity.

Each of these physiologic stressors are found in many chronic inflammatory diseases and have been shown to be reduced by the polyphenolic compound, resveratrol, 3,5,4′-trihydroxystilbene, as either the trans form or a cis/trans mixture.

### RESVERATROL

Resveratrol 12 is a major component of nutritional supplement 10. With reference to FIG. 1 a simplified pathway is shown where resveratrol 12 acts in cells and tissues of the body to reduce the physiologic stressors listed above, acting in large part by inducing the SIRT1 gene referenced by number 18, and thereby produces increased amounts of an enzyme sirtuin 1 referenced by number 20.

In the instant embodiment, resveratrol 12 is a natural extract in a purified form. Natural sources of resveratrol include wine, grape skins, peanuts, cocoa, Japanese knotweed, blueberries, and cranberries; among others,

In some examples, synthetic resveratrol is used in conjunction with the other components of supplement 10. In yet other examples the supplement comprises a mixture of naturally derived and synthetically derived resveratrol in nutritionally effective amounts relative to a given target physiologic stress.

Although it is linked to the reduction of numerous chronic inflammation symptoms, resveratrol 12 alone is insufficient to adequately address and overcome a given physiologic stress. Activity of sirtuin 1 is highly dependent on the levels of a coenzyme nicotinamide adenine dinucleotide (NAD+) referenced by number 26 in FIG. 1. Sirtuin 1 shows relatively little activity if the cells and tissues involved are depleted of NAD+.

Many of these chronic inflammatory diseases are characterized by high levels of poly (ADP-ribose) polymerase/synthetase activity (PARP or PARS), due to the stimulation of this enzyme by the action of peroxynitrite on the DNA in the nuclei of these cells. Furthermore, high level PARP can lead to massive depletion of NAD+ in cells, because NAD+ is the substrate for the PARP enzyme. Consequently, such high level PARP activity may largely prevent potentially favorable responses to resveratrol in such chronic inflammatory diseases.

Importantly, supplement 10 is a combination of nutritional ingredients, which provides not only resveratrol 12 (either trans or cis/trans), but also two other agents which are needed to restore NAD+ pools in cells impacted by such inflammatory diseases. Therefore, supplement 10 restores the enzymatic activity of Sirt1.

As shown in FIG. 1, in addition to resveratrol 12, NAD+ formation, referenced at number 26, requires nicotinic acid or a nicotinic acid precursor 14, and D-ribose 16.

### NICOTINIC ACID OR NICOTINIC ACID PRECURSOR

The vitamin Niacin provides metabolites required for NAD+ formation. Accordingly, supplements described herein contain relatively high doses of the nicotinic acid form of niacin, or a nicotinic acid precursor, such as inositol hexanicotinate, commonly called “low-flush” or “no-flush” niacin.

The dosage of nicotinic acid or nicotinic acid precursor present in a given preparation or present in a given treatment regimen is selectable relative to the target physiologic stressor. In one example, a standard RDA dosage of approximately 11-18 mg daily is used. In another example, a high dose of 50 mg is used. In yet another example, a very high dose of greater than 1 g is appropriate.

in the present embodiment, supplement 10 contains conventional nicotinic acid, in certain examples, more slowly metabolized forms of niacin are selected for a timed-release of nicotinic acid into the organism. Rather than providing readily assimilated nicotinic acid to the organism, certain examples rely on niacin forms that must first be converted to nicotinic acid so that the release of nicotinic acid is sustained over a period of time.

The nicotinamide form of niacin, however, is not to be included here because nicotinamide inhibits Sirt1 activity and thus may be counterproductive.

In order for nicotinic acid or nicotinic acid precursor 14 to elevate available NAD+ pools and its precursor nicotinamide mononucleotide (NMN), it must react in the cellular environment with phosphoribosylpyrophosphate (PRPP). To some extent, PRPP can become rate limiting in this process.

### D-RIBOSE

In order to maintain NAD+ production and overcome the rate limiting effects of PRPP, another component able to drive PRPP supplies is needed. The sugar D-ribose acts as a precursor for PRPP, raising its levels in cells.

D-ribose has been fairly widely used as a nutritional supplement to restore adenosine 5′-triphosphate (ATP) pools in cells and tissues depleted of adenine nucleotides including ATP. Here, the inventor utilizes D-ribose for an altogether different purpose; to restore NAD+ pools, not ATP.

With reference to FIG. 1 supplement 10 is shown to include D-ribose. In the instant example, it is the role of D-ribose to drive PRPP supply and consequently allow nicotinic acid to restore NAD+ pools. With the now sufficient NAD+ levels and ample nicotinic acid and D-ribose to sustain those NAD+ levels, the capacity for resveratrol to increase sirtuin 1 enzymatic activity and thereby reduce target physiologic stressors is greatly enhanced.

### PREPARATIONS

As shown in FIGS. 2-4, supplement 10 can be introduced to a given organism through various preparations containing resveratrol 12, nicotinic acid or nicotinic acid precursor 14, and D-ribose 16. These preparations are represented by way of example only in single preparation 11, preparation pair 13, and separate preparations 15.

Each of FIGS. 2-4 represents only a sample preparation for introduction of supplement 10 into a given organism. A preparation defines the vehicle for transporting the novel combination of ingredients of supplement 10 into an organism,

Turning our attention specifically to FIG. 2, single preparation 11 is now described. Single preparation 11 defines a capsule 50 that contains nutritionally effective amounts of resveratrol 12, nicotinic acid or nicotinic acid precursor 14, and D-ribose 16. In the instant example, supplement 10 is wholly contained within single preparation 11. Additionally, single preparation 11 contains an excipient 40 and is administered via a delivery method 64 to an organism 60.

In this example capsule 50 is a standard two-piece capsule as known in the art. In various examples these capsules are made from soft shell or hard shell capsule ingredients such as animal and plant gelatin proteins.

Often, nutritional supplement preparations contain peripheral ingredients known as excipients. By way of example, excipient 40 represents any number or combination of ingredients added to a given preparation for consistency, flavor, preservation, look, feel, consumability, and timed-release, among others.

Single preparation 11 is administered via delivery method 64. In this example, delivery method 64 is an oral administration of the supplement-containing preparation. In another example, the delivery method is buccal delivery, where the supplement containing preparation is not consumed. In yet another example, the delivery method is a topical cream applied to the organism.

Turning our attention now to FIGS. 3 and 4, we see that the individual ingredients of supplement 10 can be administered in separate preparations. Preparation pair 13, for example, includes a preparation in the form of a tablet 51 and a preparation in the form of a powder 52. In this example, resveratrol 12 is contained in tablet form and the nicotinic acid or nicotinic acid precursor and D-ribose are contained in powder 52.

FIG. 3 exemplifies the many preparation types and preparation excipients that can be used to contain and deliver supplement 10. In the example shown in FIG. 3, tablet 51 for instance, contains an excipient binder 42. A binder is used to maintain the structure of the tablet. Powder 52 also contains an excipient, disintegrant 44. A disintegrant is used in preparations to cause them to uniformally disintegrate when consumed or mixed into a liquid.

In this example the organism in question is a human 62. Although humans are a primary organism for which supplement 10 may be useful, any organism susceptible to chronic inflammation and other physiologic stressors described herein may constitute a target organism. Certain household pets, farm animals, or livestock, for instance, are susceptible to the inflammatory maladies described above. The combination embodied in supplement 10 and the relevant treatments claimed here are, therefore, equally applicable to non-human subjects.

In a further alternative example, supplement 10 is divided into multiple preparations, each preparation containing only a single component of the supplement, either resveratrol 12, nicotinic acid or nicotinic acid precursor 14, and D-ribose 16. In such examples where supplement 10 is delivered in multiple preparations, it becomes important that the individual preparations are delivered contemporaneously.

If for instance a preparation of resveratrol 12 is delivered and is metabolized before the balance of supplement 10 can be delivered, the effects of the supplement are greatly reduced.

The time-frame within which a delivery can be considered contemporaneous, is a function of the rate at which components are metabolized and remain bioavailable to the organism in the appropriate form. In a preferred embodiment, all of the components of supplement 10 are delivered at the same time.

Just as FIG. 3 showed the various preparation combinations achievable for delivery of supplement 10, FIG. 4 provides examples of yet other combinations as separate preparations 15. Separate preparations 15 include, a first preparation defining a liquid suspension 53, a second preparation defining a supplemented food 54, and a third preparation defining a supplemented drink 55.

Liquid suspension 53 represents a standard liquid concentration of a given component of supplement 10. Liquid suspensions are well known in the art and constitute a viable preparation for supplement 10. Liquid suspension 53 further includes an excipient preservative 46 as known in the art.

Supplemented-food 54 represents a number of supplementable foods capable of defining a preparation for supplement 10. Examples of supplemented foods include chocolates, chewables, chewing gum, and other foodstuffs containing nutritionally effective amounts of a given component of supplement 10.

By way of example, supplemented-food 54 further includes an excipient flavor 48. Excipient flavors are used widely in the relevant art to make supplements more palatable for buccal and oral delivery.

In this example supplemented food 54 is a preparation configured to deliver nicotinic acid or nicotinic acid precursor 14. In another example, the supplemented food is used to deliver an alternative component of supplement 10.

Lastly, supplemented-drink 55 is a preparation used in the example shown in FIG. 4, to contain D-ribose. Supplemented-drink 55 is in the form of a drink for oral ingestion. Supplemented drinks are known in the art to provide a preparation type able to contain nutritionally effective amounts of a given nutritional supplement. In this example, D-ribose is mixed into an excipient liquid. In various examples the excipient liquid is water, juice, or any liquid capable of containing effective amounts of D-ribose.

The possible combinations of preparations, excipient ingredients, and delivery-methods described above all culminate in the contemporaneous delivery of all ingredients of supplement 10 to an organism.

Importantly, methods of treatments of relevant physiologic stressors are also varied. Contemplated methods of treatments involve the delivery of supplement 10 according to the above described preparations in nutritionally effective amounts.

Nutritionally effective amounts are largely a function of the target physiologic stressor and its relative degree of intensity in the organism. In a first example, nutritionally effective amounts are selectable based upon this function.

In a second example, more broadly applicable to the needs of the general populous involves treatment with the recommended daily amounts, as known in the art, of each of resveratrol 12, nicotinic acid or nicotinic acid precursor 14, and D-ribose 16.

In a third treatment example, also pertinent to the needs of the general populous, amounts in excess of the recommended daily allowance as known in the art are used.

In this example, nutritionally effective amounts include the combination of ingredients introduced at least once daily where the resveratrol dose is at least 30 mg per day; the nicotinic acid or nicotinic acid precursor dose is at least 30 mg per day; and the D-ribose dose is at least 3 grams per day. In various other examples, the relative nutritionally effective amounts exceed those of the instant example.

Also contemplated, are treatments including high doses of resveratrol 12 and nicotinic acid 14, but only standard RDA doses or even less of D-ribose 16. If PRPP stores are otherwise adequate, this varied combination of supplement 10 can still drive NAD+ production and therefore sustain the sirtuin 1 enzymatic activity.

By way of example, a treatment preparation containing 50 mg of resveratrol and 200 to 500 mg of nicotinic acid as inositol hexanicotinate, with little or no D-ribose, taken one to three times per day may also provide adequate NAD+ levels for sustaining the sirtuin 1 activity.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A nutritional supplement, comprising;
a combination of resveratrol, nicotinic acid or a nicotinic acid precursor, and D-ribose in nutritionally effective amounts; and
wherein the combination is configured to increase Sirtuin 1 enzymatic activity and to initiate increased NAD+ production for sustaining said Sirtuin 1 enzymatic activity.

2. The nutritional supplement of claim 1, wherein the nicotinic acid precursor is inositol hexanicotinate.

3. The nutritional supplement of claim 1, wherein the resveratrol is cis-Resveratrol.

4. The nutritional supplement of claim 1, wherein the resveratrol is a mixture of cis-Resveratrol and trans-Resveratrol.

5. The nutritional supplement of claim 1, further comprising an excipient.

6. The nutritional supplement of claim 5, wherein the excipient is a binder.

7. The nutritional supplement of claim 5, wherein the excipient is a disintegrant.

8. A nutritional supplement, comprising:
a combination of resveratrol, nicotinic acid or a nicotinic acid precursor, and D-ribose in nutritionally effective amounts;
wherein:
the resveratrol increases Sirtuin 1 enzyme production;
the nicotinic acid or nicotinic acid precursor and the D-ribose stimulate cellular NAD+ production; and
the combination of ingredients increases Sirtuin 1 enzymatic activity thereby inhibiting a detrimental physiological stress.

9. The nutritional supplement of claim 8, wherein the nicotinic acid precursor is selected for prolonged nicotinic acid production.

10. The nutritional supplement of claim 9, wherein the precursor type is inositol hexanicotinate.

11. The nutritional supplement of claim 8, wherein the combination includes:
1 part resveratrol;
1 part nicotinic acid or nicotinic acid precursor; and
98 parts D-ribose.

12. The nutritional supplement of claim 8, wherein the detrimental physiological stress includes oxidative stress.

13. The nutritional supplement of claim 8, further including an excipient.

14. The nutritional supplement of claim 8, further including a preservative.

15. The nutritional supplement of claim 8, further including a flavor.

16. A method of treating a physiological stress, comprising introducing into an organism a combination of ingredients consisting essentially of resveratrol, nicotinic acid or a nicotinic acid precursor, and D-ribose in nutritionally effective amounts.

17. The treatment method of claim 16, wherein the ingredients are introduced via an oral capsule.

18. The treatment method of claim 16, wherein the organism is a human.

19. The treatment method of claim 16, wherein the physiological stress is chronic inflammation.

20. The treatment method of claim 16, wherein
the combination of ingredients are introduced at least once daily;
the resveratrol dose is at least 30 mg per day;
the nicotinic acid or nicotinic acid precursor dose is at least 30 mg per day; and
the D-ribose dose is at least 3 grams per day.
